(19) United States
(12) Patent Application Publication
(10) Pub. No.: US 2024/0009304 A1
(43) Pub. Date: Jan. 11, 2024

(54) USE OF NGF ANTIBODY IN CIPN PAIN

(71) Applicants: Dartsbio Pharmaceuticals Ltd., Zhongshan, Guangdong (CN); Shanghai Mabstone Biotechnology Co., Ltd., Shanghai (CN)

(72) Inventors: Chunhe WANG, Zhongshan, Guangdong (CN); Zuobin XIE, Zhongshan, Guangdong (CN); Huaping DING, Zhongshan, Guangdong (CN); Yili CHEN, Zhongshan, Guangdong (CN); Huanhuan LI, Zhongshan, Guangdong (CN); Lei TANG, Zhongshan, Guangdong (CN)

(73) Assignees: Dartsbio Pharmaceuticals Ltd., Zhongshan, Guangdong (CN); Shanghai Mabstone Biotechnology Co., Ltd., Shanghai (CN)

(21) Appl. No.: 18/036,548

(22) PCT Filed: Nov. 10, 2021
(86) PCT No.: PCT/CN2021/129914

§ 371 Date: May 11, 2023

(30) Foreign Application Priority Data
Nov. 12, 2020 (CN) 202011263674.2

(51) Int. Cl. A61K 39/395; C07K 16/22; A61K 31/337; A61K 31/475; A61K 33/243; A61P 25/02

(52) U.S. Cl. CPC A61K 39/3955; C07K 16/22; A61K 31/337; A61K 31/475; A61K 33/243; A61P 25/02; C07K 2317/24; A61K 2039/505

(57) ABSTRACT

Provided is use of an NGF antibody in CIPN pain. In particular, the NGF antibody blocks the interaction of nerve growth factors (NGF) with receptors thereof, and thus can treat and/or prevent chemotherapy-induced peripheral neuropathy (CIPN) pain.

### TECHNICAL FIELD

The present invention belongs to biomedical field, in particular, the present invention relates to the use of NGF antibody in CIPN pain.

### BACKGROUND

In recent years, with the incidence rate of malignant tumors increasing year by year, the use of chemotherapy drugs is also increasingly widespread. However, the pain caused by chemotherapy drugs seriously affects the life quality of patients, causing or aggravating depression, insomnia, fatigue and other symptoms, and leading to the change of chemotherapy dose and even the termination of chemotherapy. Among them, chemotherapy-induced peripheral neuropathy (CIPN) is a common adverse reaction of cancer treatment. The proportion of chemotherapy-induced peripheral neuropathy (CIPN) pain in chemotherapy patients varies from 40% to 70%, with pain, numbness and tingling as the clinical symptoms.

So far, there is no ideal drug and method that can effectively control or prevent chemotherapy-induced peripheral neuropathy (CIPN) pain without affecting the anti-tumor activity of chemotherapy drugs. At present, the drugs for the treatment of chemotherapy-induced peripheral neuropathy (CIPN) pain are mainly opioids, but their effect is not satisfied, and the use of opioids is limited. Drugs commonly used to treat neuropathic pain, such as amitriptyline, gabapentin and pregabalin, do not seem to be more effective than placebo in the treatment of CIPN pain. Duloxetine is the only non-opioid drug proven to be effective in the treatment of CIPN pain. Duloxetine is a selective 5-hydroxytryptamine (5-HT) and norepinephrine (NE) reuptake inhibitor. Preclinical studies showed that duloxetine is a neuronal 5-HT and NE reuptake strong inhibitor, and its inhibitory effect on dopamine reuptake is relatively weak. The results of in vitro study showed that duloxetine has no obvious affinity with dopaminergic receptor, adrenergic receptor, cholinergic receptor, histaminergic receptor, opioid receptor, glutamate receptor and GABA receptor. Duloxetine does not inhibit monoamine oxidase. Duloxetine has antidepressant effect in clinic, but the mechanism of its analgesic effect in CIPN is not clear.

The mechanism of CIPN pain may not be the same as the common acute and chronic pain due to the unsatisfactory effect of common analgesics in the treatment of CIPN pain. For this reason, drugs with obvious analgesic effect in other pain cannot be directly used for CIPN pain. The pain caused by chemotherapeutic drugs is not caused by a single mechanism, but is the result of the interaction of multiple factors and multiple links. The corresponding treatment is also complex, and there are many factors affecting the treatment effect. At present, although the scheme based on symptomatic treatment in clinical practice can temporarily alleviate the pain of patients with chemotherapy-induced neuropathic pain to a certain extent, further it still needs research and exploration to obtain satisfactory effect.

In conclusion, there is an urgent need in the field to develop an inhibitor that can be applied to chemotherapy-induced peripheral neuropathy (CIPN) pain. In our study, we found that blocking the binding of nerve growth factor (NGF) to its receptor with antibody can significantly reduce the pain induced by chemotherapy-induced peripheral neuropathy (CIPN), which mechanism is completely different from the pharmacological mechanism of known duloxetine.

### CONTENT OF THE INVENTION

An object of the present invention is to provide a humanized recombinant monoclonal antibody targeting nerve growth factor (NGF), which can effectively inhibit chemotherapy-induced peripheral neuropathy (CIPN) pain.

In the first aspect of the present invention, there is provided the use of a monoclonal antibody targeting nerve growth factor for the preparation of a drug for treating and/or preventing chemotherapy-induced peripheral neuropathic pain, wherein the chemotherapy-induced peripheral neuropathy is caused by a chemotherapeutic agent.

In another preferred embodiment, the treatment of chemotherapy-induced peripheral neuropathic pain with regular analgesics is ineffective, wherein the regular analgesics are selected from the group consisting of morphine, cannabis, tetrahydrocannabinone and derivatives, dolantin, fentanyl, codeine, hydrocodone methoxynaphthalene propionic acid (naproxen), aspirin, amitriptyline, gabapentin, paracetamol, diclofenac, ibuprofen, duloxetine or pregabalin and the like and non-steroidal anti-inflammatory and analgesic drugs.

In another preferred embodiment, the monoclonal antibody is a humanized recombinant monoclonal antibody.

In another preferred embodiment, the heavy chain variable region of the monoclonal antibody targeting nerve growth factor has the amino acid sequence shown in SEQ ID No: 1.

In another preferred embodiment, the light chain variable region of the monoclonal antibody targeting nerve growth factor has the amino acid sequence shown in SEQ ID No: 2.

In another preferred embodiment, the heavy chain variable region of the monoclonal antibody targeting nerve growth factor has the amino acid sequence shown in SEQ ID No: 1, and the light chain variable region has the amino acid sequence shown in SEQ ID No: 2.

In another preferred embodiment, the heavy chain sequence and light chain sequence of the monoclonal antibody are as shown in SEQ ID No: 3 and SEQ ID No: 4, respectively.

In another preferred embodiment, the chemotherapeutic agent is selected from the group consisting of taxanes, platinums, vinca alkaloids, gemcitabine, bortezomib, thalidomide, vinorelbine, or combinations thereof.

In another preferred embodiment, the chemotherapeutic agent is selected from the group consisting of paclitaxel, cisplatin, vincristine, or combinations thereof.

In another preferred embodiment, the one or more chemotherapeutic agents are used for the treatment of cancer.

In another preferred embodiment, the cancer is selected from ovarian cancer, cervical cancer, colorectal cancer, prostate cancer, breast cancer, testicular cancer, leukemia, neuroblastoma, Hodgkin lymphoma, non Hodgkin lymphoma and non-small cell lung cancer.

In another preferred embodiment, the monoclonal antibody targeting nerve growth factor is administered before, during or after the administration of the chemotherapeutic agent.

In another preferred embodiment, the dose of the monoclonal antibody targeting nerve growth factor is 50-2000 mg/50 kg.

In another preferred embodiment, the monoclonal antibody targeting nerve growth factor significantly increases the threshold of CIPN pain.

In another preferred embodiment, the monoclonal antibody targeting nerve growth factor significantly improves the CIPN pain score.

In the second aspect of the present invention, there is provided a pharmaceutical composition comprising i) one or more monoclonal antibodies targeting nerve growth factor;
ii) one or more of the chemotherapeutic agents; and
iii) a pharmaceutically acceptable carrier.

In another preferred embodiment, the monoclonal antibody targeting nerve growth factor in the pharmaceutical composition is DS002.

In another preferred embodiment, the pharmaceutical composition may also include other drugs for treating and/or preventing chemotherapy-induced peripheral neuropathy, and the other drugs for treating and/or preventing chemotherapy-induced peripheral neuropathy include small molecule drugs (such as CXCR2 inhibitors, PARP inhibitors, etc.).

In another preferred embodiment, the pharmaceutical composition is an injection.

In another preferred embodiment, the monoclonal antibody targeting nerve growth factor or the pharmaceutical composition containing the same is administered 1-5 times every 3 days, every 4 days, every 5 days, every 6 days, every 10 days, and every 2 weeks during the treatment period, wherein the treatment period is 6 days, 1 week, 2 weeks, 3 weeks, 4 weeks, 5 weeks, or 6 weeks.

In another aspect, there is provided a method of treating and/or preventing chemotherapy-induced peripheral neuropathic pain, the method comprising administering a therapeutically effective amount of a monoclonal antibody targeting nerve growth factor to a subject in need of treatment.

In a third aspect of the present invention, there is provided a kit of a drug for treating and/or preventing chemotherapy-induced peripheral neuropathic pain, the kit comprises a container in which a monoclonal antibody targeting nerve growth factor or a pharmaceutical composition comprising the same is contained; and a label or instruction that indicates that the kit is used to treat and/or prevent chemotherapy-induced peripheral neuropathic pain.

In another preferred embodiment, the kit further comprises a companion diagnostic reagent, which is a reagent for detecting NGF.

In another preferred embodiment, the diagnostic reagent is used to detect the quantity and activity, etc., of NGF.

In a fourth aspect of the present invention, there is provided a use of the kit according to the third aspect for treating and/or preventing chemotherapy-induced peripheral neuropathic pain.

It should be understood that within the scope of the present invention, the above technical features of the present invention and the technical features described in detail below (as in the Examples) can be combined with each other to form new or preferred technical solutions, which are omitted here.

### BRIEF DESCRIPTION OF THE DRAWINGS

FIG. 1 is a flowchart showing the administration to a paclitaxel induced rat neuropathic pain model.

FIG. 2 is a histogram showing changes of mechanical paw withdrawal threshold of animals in each group during the paclitaxel induced model test.

FIG. 3 is a graph showing changes of mechanical paw withdrawal threshold of animals in each group during the paclitaxel induced model test.

FIG. 4 shows score changes of acetone induced paw withdrawal response of animals in each group during the paclitaxel induced model test.

FIG. 5 shows the response frequency of cold allodynia of animals in each group during the paclitaxel induced model test.

FIG. 6 is a histogram showing changes of mechanical paw withdrawal threshold of animals in each group during the vincristine induced model test.

FIG. 7 is a graph showing changes of mechanical paw withdrawal threshold of animals in each group during the vincristine induced model test.

FIG. 8 shows the score changes of acetone induced paw withdrawal response of animals in each group during the vincristine induced model test.

FIG. 9 shows the response frequency of cold allodynia of animals in each group during the vincristine induced model test.

FIG. 10 is a histogram showing changes of mechanical paw withdrawal threshold of animals in each group during the cisplatin induced model test.

FIG. 11 is a graph showing changes of mechanical paw withdrawal threshold of animals in each group during the cisplatin induced model test.

FIG. 12 shows the score changes of acetone induced paw withdrawal response of animals in each group during the cisplatin induced model test.

FIG. 13 shows the response frequency of cold allodynia of animals in each group during the cisplatin induced model test.

FIG. 14 shows the amino acid sequence of the humanized recombinant monoclonal antibody DS002.

### EMBODIMENTS

After extensive and in-depth research, the inventor of the present invention unexpectedly found firstly that the humanized recombinant monoclonal antibody targeting nerve growth factor (such as DS002) can effectively inhibit chemotherapy-induced peripheral neuropathic (CIPN) pain with low side effects. The experiment shows that it is unexpected that conventional or potent analgesics cannot effectively relieve CIPN pain caused by chemotherapy agents (such as taxanes, platinums and vincristines), however, the antibody of the present invention (such as DS002) can effectively relieve these refractory or intractable CIPN pain. On this basis, the inventor completed the present invention.

### Terms

As used herein, the term “treatment” refers to any treatment of a disease and/or disorder in an animal, particularly a human, including: (i) inhibiting the disease and/or disorder, i.e. preventing its progression; (ii) alleviating the disease and/or symptom, i.e., leading to regression of the disease and/or symptom. For example, treatment of CIPN pain includes preventing or alleviating CIPN pain, or relieving or alleviating a symptom of CIPN pain.

As used herein, the term “prevention” refers to (i) preventing the development of a disease and/or disorder; and/or (ii) preventing the disease and/or disorder from getting worse in a state where the disease and/or disorder has already developed.

Chemotherapy-Induced Peripheral Neuropathy (CIPN)

As used herein, “chemotherapy-induced peripheral neuropathy (CIPN)” refers primarily to a group of dose-dependent tumor chemotherapeutic drugs-induced peripheral neuropathy. The drugs that cause CIPN mainly include platinum chemotherapy drugs, anti-tubulin drugs, suramin sodium, thalidomide (TLD), epothilone and bortezomib, and the like. The incidence of CIPN mainly depends on the type of drug selected and the duration of use. CIPN affects the life of patients with breast cancer, colorectal cancer, testicular cancer and hematopoietic malignancies after chemotherapy. The most common symptoms of CIPN are pain (which may be constant or intermittent, such as flashing pain or stabbing pain), burning, tingling (“pins and needles” or electric/shock-like pain), anesthesia (it can be numbness or reduced ability to feel pressure, touch, heat or cold) etc.

Chemotherapeutic Agent

“Chemotherapeutic agent” or “antineoplastic agent” refers to an agent that can reduce, prevent and/or delay metastasis or growth of tumor, or kill tumor cells by causing necrosis or apoptosis of tumor cells by administrating drugs in an effective amount, so as to reduce, prevent and/or delay the metastasis or growth of a tumor in a subject with a neoplastic disease. Chemotherapy is currently one of the most effective means of treating cancer, and chemotherapy, surgery and radiotherapy are called the three major cancer treatments. Surgery and radiotherapy, which are local treatments, are only effective for the tumor at the treatment site, but can't achieve effective treatment for potential metastatic lesions (cancer cells have actually metastasized, which is hard to be discovered and detected clinically due to the limitations of current technical means) and clinically metastatic cancer. Chemotherapy is a means of systemic treatment. No matter what route of administration is adopted (oral, intravenous and body cavity administration, etc.), chemotherapy drugs will be delivered to most organs and tissues of the body along with blood circulation. Therefore, chemotherapy is the main treatment method for some tumors with a tendency of systemic dissemination and intermediate and advanced tumors that have metastasized.

The chemotherapeutic agents of the present invention include taxanes (paclitaxel), platinums (e.g., cisplatin, carboplatin, oxaliplatin), vinca alkaloids (vincristine), thalidomide, and the like.

Paclitaxel is an anticancer drug extracted from taxane, which mainly inhibits the malignant proliferation of tumor cells by promoting the polymerization of intracellular tubulin, maintaining the stability of tubulin, and inhibiting cell mitosis. Paclitaxel is widely used in the treatment of solid tumors, and two serious adverse reactions after paclitaxel chemotherapy are myelosuppression and neuropathic pain. Neuropathic pain caused by paclitaxel mainly manifests as peripheral sensory hyperalgesia, burning pain, irritation and numbness and other symptoms. The symptom can last for several months to several years after paclitaxel is no more administered and is an intractable pain with no effective treatment currently. Some cancer patients treated with paclitaxel even had to discontinue treatment due to severe pain.

It has been reported in the literature that a model of peripheral neuropathic pain in SD rats can be successfully established by intermittent and repeated intraperitoneal injection of paclitaxel, which is shown as sciatic nerve myelin sheath swelling, partial myelin sheath vacuolation, partial Schwann cell structure destruction, and Schwann cell nuclei increase in tissue structure, and decreased mechanical pain threshold.

Humanized Recombinant Monoclonal Antibody Targeting Nerve Growth Factor

Targeted nerve growth factor (Nerve growth factor, NGF) belongs to the family of neurotrophic factors, originally extracted from mouse submandibular gland and snake venom, and exists in almost all vertebrates. NGF exists in different multimers (precursors) in different species, in which the 0 subunit has complete biological activity of NGF and is called R-NGF; the mature free P-NGF consists of two 118 amino acid polypeptides formed by non-covalent bonds. Current research suggests that in embryos and juvenile animals, NGF promotes the growth, differentiation and loss repair of peripheral sensory and sympathetic neurons. In adult animals, NGF mainly regulates inflammatory responses and sensitizes nociceptors in the event of injury and inflammation. After NGF binds to the NGF functional receptor TrkA on the surface of nociceptors, it activates signaling pathways such as cytoplasmic ERK and PLC/PKC, reduces the threshold of neuronal action potential, increases neuronal excitability, and sensitizes pain sensation.

As used herein, the terms “antibody of the invention” or “anti-NGF antibody of the invention” are used interchangeably and refer to an antibody that specifically targets NGF, particularly human NGF. The antibody of the present invention is preferably a monoclonal antibody. The antibody can be an intact antibody or an active fragment of an antibody. It should be understood that the term also includes a single chain antibody (scFv), a nanobody. In addition, the antibody of the invention may be of animal origin (e.g., murine origin), humanized, chimeric, fully human antibody, or combinations thereof.

Preferably, the heavy chain variable region of the humanized recombinant monoclonal antibody targeting nerve growth factor has an amino acid sequence shown in SEQ ID NO: 1.

Preferably, the light chain variable region of the humanized recombinant monoclonal antibody targeting nerve growth factor has an amino acid sequence shown in SEQ ID NO:2.

Preferably, the heavy chain variable region of the humanized recombinant monoclonal antibody targeting nerve growth factor has an amino acid sequence shown in SEQ ID NO:1, and the light chain variable region has an amino acid sequence shown in SEQ ID NO:2.

(SEQ ID NO: 1)
QVQLVQSGAEVKKPGASVKVSCKASGYTFTNYLIEWVRQAPGQGLEWMGV
INPRSGATNYNEKFKDRITITKDTSKNQVVLTMTNMDPVDTATYYCARGN
YRFEAYWGQGTLVTVSS.
(SEQ ID NO: 2)
DILMTQSQKFMSTSVGDRVSITCKASQNVRTAVAWYQQKPGQSPKALILA
SNRHTGVPDRFTGSGSGTDFTLTISNMQSEDLADYFCQQYSSYPFTFGSG
TKLEIK.

As used herein, “DS002”, a humanized recombinant monoclonal antibody targeting nerve growth factor, is a multi-domain complex composed of two heavy chains and two light chains linked by disulfide bonds, it can bind to the NGF protein molecule and block the binding of the NGF protein molecule to its receptor TrkA protein molecule. DS002 structurally belongs to the IgG1 subtype of human IgG, and the light chain is of the kappa type.

Preferably, the heavy chain sequence and light chain sequence of DS002 are shown in SEQ ID NO:3 and SEQ ID NO:4

(SEQ ID NO: 3)
QVQLKESGPGLVAPSETLSITCTVSGFSLTGYGVNWVRQPPGKGLEWLGM
IWADGDTDYNSALKSRLTISKDNSKSQVFLKVNNLQTDDTARYYCARDSY
YYGYNFFDVWGAGTTVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG
K.
(SEQ ID NO: 4)
DIQMTQSPSSLSASVGDRVTISCRASQDISNYLNWYQQKPEGTLKLLIYY
TSRLHSGVPSRFSGSGSGTDYSLTISSLQQEDIATYFCQQGNTLPRTFGG
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

Preferably, the heavy chain (a) and light chain (b) amino acid sequences of DS002 are shown in FIG. 14.

In the present invention, the anti-NGF antibody can be used before, during and after the use of the chemotherapeutic agent.

In the present invention, the antibody of the present invention also includes its conservative variants, which means polypeptides formed by replacing at most 10, preferably at most 8, more preferably at most 5, most preferably at most 3 amino acids of the amino acid sequence of the antibody of the present invention with amino acids of similar or similar nature. These conservatively variant polypeptides are preferably produced by amino acid substitutions according to Table A.

TABLE A

| Original residue | Representative substitutions | Preferred substitution |
|---|---|---|
| Ala (A) | Val; Leu; Ile | Val |
| Arg (R) | Lys; Gln; Asn | Lys |
| Asn (N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro; Ala | Ala |
| His (H) | Asn; Gln; Lys; Arg | Arg |
| Ile (I) | Leu; Val; Met; Ala; Phe | Leu |
| Leu (L) | Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Leu |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala | Leu |

In the present invention, the dosage of anti-NGF antibody (such as DS002) is not particularly limited, and can be any safe and effective dosage. A representative dose may be, for example, 50-2000 mg/50 kg body weight, preferably 100-1000 mg/50 kg body weight.

Use of the Monoclonal Antibody Targeting Nerve Growth Factor

The present invention provides the use of the above-mentioned monoclonal antibody targeting nerve growth factor, for preparing a medicine or a pharmaceutical composition for treating and/or preventing chemotherapy-induced peripheral neuropathic pain, wherein the chemotherapy-induced peripheral neuropathy is caused by chemotherapeutic agents, wherein the chemotherapeutic agents are as described above.

Preferably, the amino acid sequence of the heavy chain variable region of the humanized recombinant monoclonal antibody targeting nerve growth factor is shown in SEQ ID NO:1.

Preferably, the light chain variable region of the humanized recombinant monoclonal antibody targeting nerve growth factor comprises the amino acid sequence shown in SEQ ID NO:2.

Preferably, the heavy chain variable region of the humanized recombinant monoclonal antibody targeting nerve growth factor comprises the amino acid sequence shown in SEQ ID NO:1, and the light chain variable region comprises the amino acid sequence shown in SEQ ID NO:2.

Preferably, the monoclonal antibody is DS002, wherein the heavy chain sequence and light chain sequence of DS002 are shown in SEQ ID NO:3 and SEQ ID NO:4, respectively.

Preferably, the chemotherapeutic agent is selected from the group consisting of paclitaxel, cisplatin, vincristine, or combinations thereof.

Pharmaceutical Composition

The present invention also provides a composition. In a preferred embodiment, the composition is a pharmaceutical composition, which contains the above-mentioned humanized recombinant monoclonal antibody targeting nerve growth factor (preferably DS002), and a pharmaceutically acceptable carrier. Generally, these materials can be formulated in a non-toxic, inert and pharmaceutically acceptable aqueous medium usually at a pH of about 5-8, preferably about 6-8, although the pH may vary depending on the nature of the formulation material and the symptom to be treated.

The formulated pharmaceutical compositions can be administered by conventional routes including, but not limited to, intratumoral, intraperitoneal, intravenous, or topical administration. The injection administration preferably includes intravenous injection, intramuscular injection, intraperitoneal injection, intradermal injection or subcutaneous injection. The pharmaceutical composition is in various conventional dosage forms in the art, preferably in the form of solid, semi-solid or liquid, it can be an aqueous solution, a non-aqueous solution or a suspension, more preferably a tablet, capsule, granule, injection or infusion, etc.

The pharmaceutical composition of the present invention is a pharmaceutical composition for preventing and/or treating pain caused by a) chemotherapy-induced peripheral neuropathy (CIPN).

The pharmaceutical composition of the present invention can be directly used to bind NGF protein molecule and block the binding of NGF protein molecule to its receptor TrkA protein molecule, so it can be used to prevent and treat pain caused by chemotherapy-induced peripheral neuropathy (CIPN).

The pharmaceutical composition of the present invention contains a safe and effective amount (such as 0.001-99 wt %, preferably 0.01-90 wt %, more preferably 0.1-80 wt %) of the above-mentioned monoclonal antibody targeting nerve growth factor of the present invention (such as DS002) and a pharmaceutically acceptable carrier or excipient. Such carriers include, but are not limited to, saline, buffers, glucose, water, glycerol, ethanol, and combinations thereof. The pharmaceutical formulation should be compatible with the mode of administration. The pharmaceutical composition of the present invention can be prepared in the form of injection, for example, prepared by conventional methods with physiological saline or an aqueous solution containing glucose and other adjuvants. Pharmaceutical compositions such as injections and solutions are preferably manufactured under sterile conditions. The administration amount of the active ingredient is a therapeutically effective amount, e.g., about 1 microgram/kg body weight to about 5 mg/kg body weight per day. In addition, the polypeptide of the present invention may also be used with other therapeutic agents.

In the present invention, preferably, the pharmaceutical composition of the present invention further comprises one or more pharmaceutically acceptable carriers. The pharmaceutical carrier is a conventional pharmaceutical carrier in the art, and the pharmaceutical carrier can be any suitable physiologically or pharmaceutically acceptable pharmaceutical adjuvant. The pharmaceutical excipient is a conventional pharmaceutical excipient in the art, preferably including pharmaceutically acceptable excipients, fillers or diluents and the like. More preferably, the pharmaceutical composition contains 0.01-99.99% of the antibody of the present invention and 0.01-99.99% of a pharmaceutical carrier, and the percentage is the mass percentage of the pharmaceutical composition.

In the present invention, preferably, the administration dose of the pharmaceutical composition is an effective amount, and the effective amount is an amount capable of alleviating or delaying the progression of a disease, degenerative or damaging symptom. The effective amount can be determined on an individual basis and will be based in part on consideration of the symptoms to be treated and the expected results. An effective amount can be determined by one skilled in the art by considering the above-mentioned factors on an individual basis and the like via routine experiments.

When the pharmaceutical composition is used, a safe and effective amount of the immunoconjugate is administered to a mammal, wherein the safe and effective amount is generally at least about 10 micrograms/kg body weight, and in most cases no more than about 50 mg/kg body weight, preferably the dose is about 100 micrograms/kg body weight to about 20 mg/kg body weight. Of course, the specific dosage should also be determined by considering the route of administration, the patient's health status and other factors, which are all within the skill of the skilled physician.

The present invention has the following main advantages:
(a) The antibody of the present invention (e.g. DS002) can significantly increase the pain threshold of chemotherapy-induced peripheral neuropathy (CIPN).
(b) The antibody of the invention (e.g. DS002) has less side effects in the treatment and/or prevention of chemotherapy-induced peripheral neuropathic (CIPN) pain.

The present invention will be further described below with reference to specific examples. It should be understood that these examples are only used to illustrate the present invention and not to limit the scope of the present invention. The following examples, of which the experimental method are not specified with detailed conditions, were usually carried out by according to normal conditions (conditions described in Sambrook et al., Molecular cloning: Laboratory manual (New York: Cold Spring Harbor Laboratory Press, 1989)) or according to manufacture conditions recommended by the manufacturer. Percentages and parts are weight percentages and weight by parts unless otherwise specified.

### EXAMPLE

Experimental Method

All female SD rats were measured on day −5 (D-5) and day −4 (D-4) for basal values of von Frey mechanical allodynia and acetone cold allodynia (the day on formally establishing the model with paclitaxel administration was recorded as DO). SD rats were randomly divided into 5 groups according to body weight, which were blank control group, vehicle control (Vehicle control), s.c., D-3/D3/D9 group, DS002 0.1 mg/kg, s.c., D-3/D3/D9 group, DS002 0.5 mg/kg, s.c., D-3/D3/D9 group, DS002 2.5 mg/kg, s.c., D-3/D3/D9 group, respectively. Except for the blank control group, the other groups of animals were injected intraperitoneally with a chemotherapeutic agent solution on DO, D2, D4 and D6, respectively, to establish a chemotherapeutic agent-induced neuropathic pain model in SD rats. Wherein, von Frey is a stimulating material used in the measurement method of mechanical stimulation pain threshold; acetone is a stimulating substance used in the measurement method of cold pain threshold.

Except for the blank control group, the other four groups were administered subcutaneously with DS002 on D-3, D3 and D9, respectively, and the von Frey echanical allodynia and acetone cold allodynia were measured on D7, D14 and D8, D15, respectively. The mechanical paw withdrawal threshold and the score and frequency of acetone-induced foot withdrawal behavior at the corresponding time point were recorded (for testing and calculation methods, see Yoon et al., pain, 59(3), 369-376. & Flatters et al., pain, 2004, 109 (1-2):150-161).

### Example 1 Pharmacodynamic Experiment of D5002 on Paclitaxel-Induced Neuropathic Pain in Rats

1. Establishment of Paclitaxel-Induced Neuropathic Pain Model in Rats

The above experimental method was used, except for the blank control group, the other groups of animals were injected intraperitoneally with a chemotherapeutic agent solution (4 mg/kg) on DO, D2, D4 and D6, respectively, to establish a paclitaxel-induced neuropathic pain model in SD rats, namely the CIPN pain model. The experimental scheme is shown in FIG. 1.

2. Experimental Results

2.1 Preventive Effect on Mechanical Allodynia

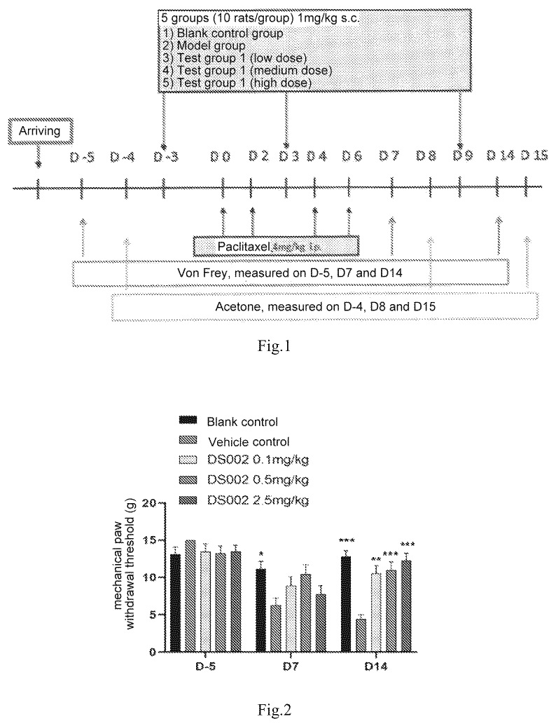

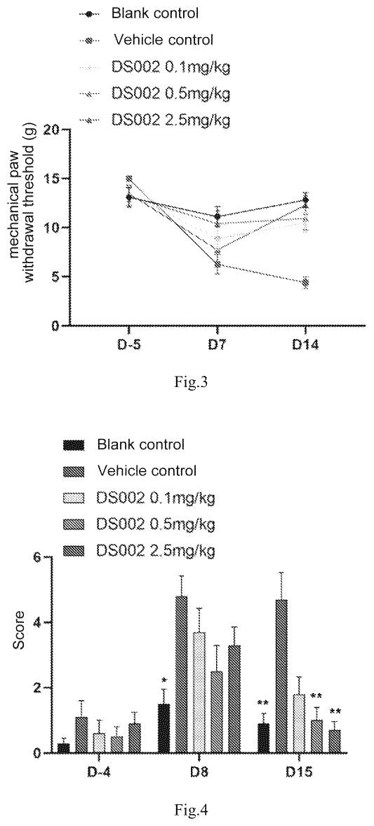

During the test, the histogram and graph of the mechanical paw withdrawal threshold of each group of animals are shown in FIG. 2 (compared with the threshold of the vehicle control group, *0.01<P<0.05; **P<0.01; ***<0.001) and FIG. 3, respectively, and the mean values of the thresholds are shown in Table 1.

| | | Mean value of mechanical paw withdrawal threshold | | |
|---|---|---|---|---|
| | | (g) ± S.E | | |
| | | 2020 Apr. 17 | 2020 Apr. 29 | 2020 May 6 |
| Group | | D-5 | D7 | D14 |
| Blank control | Mean ± S.E | 13.9 ± 0.98 | 11.13 ± 1.03* | 12.83 ± 0.75*** |
| Vehicle control, | Mean ± S.E | 15.00 ± 0.00 | 6.28 ± 0.99 | 4.40 ± 0.61 |
| s.c., D-3/D3/D9 | | | | |
| DS002-0.1 mg/kg, | Mean ± S.E | 13.48 ± 1.03 | 8.90 ± 1.20 | 10.55 ± 1.04** |
| s.c., D-3/D3/D9 | | | | |
| DS002 · 0.5 mg/kg, | Mean ± S.E | 13.25 ± 0.98 | 10.40 ± 1.33 | 10.94 ± 1.16*** |
| s.c., D-3/D3/09 | | | | |
| DS002 · 2.5 mg/kg, | Mean ± S.E | 13.50 ± 0.82 | 7.74 ± 1.19 | 12.30 ± 0.94*** |
| s.c., D-3/D3/09 | | | | |

Note:
1. The day on formally establishing the model with paclitaxel administration was recorded as D0;
2. Compared with vehicle control group, “*” 0.01 < P < 0.05, “**” P < 0.01; “***” P < 0.001.

The results showed that: compared with the Vehicle control, after injection of the agent DS002 of the present invention, the mechanical paw withdrawal threshold of rats was significantly increased.

2.2 Preventive Effect on Cold Allodynia

During the test, the histogram of acetone-induced foot withdrawal response score and the histogram of the response frequency of cold allodynia in each group of animals are shown in FIG. 4 (compared with the threshold of the vehicle control group, *0.01<P<0.05; **P<0.01) and FIG. 5 (compared with the threshold of the vehicle control group, *0.01<P<0.05; **P<0.01; ***<0.001), respectively, the mean value of the total score and the mean value of the response frequency are shown in Table 2.

TABLE 2
Mean values of acetone-induced foot withdrawal response score and the response
frequency of cold allodynia in each group of animals during the test

| | | 2020 | | 2020 | | 2020 | |
|---|---|---|---|---|---|---|---|
| | | Apr. 18 | | Apr. 30 | | May 7 | |
| | | D-4 | | D 8 | | D 15 | |
| Group | | Score | frequency | Score | frequency | Score | frequency |
| Blank | Mean ± | 0.30 ± | 9.90 ± | 1.50 ± | 33.30 ± | 0.90 ± | 16.50 ± |
| control | S.E | 0.15 | 5.04% | 0.45* | 8.65%** | 0.31** | 5.50%** |
| Vehicle | Mean ± | 1.10 ± | 23.20 ± | 4.80 ± | 83.40 ± | 4.70 ± | 73.40 ± |
| control, | S.E | 0.50 | 9.99% | 0.63 | 7.45% | 0.83 | 8.34% |
| s.c., | | | | | | | |
| D-3/D | | | | | | | |
| 3/D 9 | | | | | | | |
| DS002-0.1 | Mean ± | 0.60 ± | 13.40 ± | 3.70 ± | 63.50 ± | 1.80 ± | 33.40 ± |
| mg/kg, | S.E | 0.40 | 8.93% | 0.73 | 9.25% | 0.53 | 9.99% |
| s.c,, | | | | | | | |
| D-3/D | | | | | | | |
| 3/D 9 | | | | | | | |
| DS002-0.5 | Mean ± | 0.50 ± | 13.30 ± | 2.50 ± | 50.10 ± | 1.00 ± | 23.20 ± |
| mg/kg, | S.E | 0.31 | 7.38% | 0.79 | 14.29% | 0.39** | 7.11%*** |
| s.c., | | | | | | | |
| D-3/D | | | | | | | |
| 3/D 9 | | | | | | | |
| DS002-2.5 | Mean ± | 0.90 ± | 19.90 ± | 3.30 ± | 70.0 ± | 0.70 ± | 19.90 ± |
| mg/kg, | S.E | 0.35 | 7.37% | 0.56 | 9.22% | 0.26** | 7.37%*** |
| s.c., | | | | | | | |
| D-3/D | | | | | | | |
| 3/D 9 | | | | | | | |

Note:
1. The day on formally establishing the model with paclitaxel administration was recorded as D 0;
2. Compared with vehicle control group, “*” 0.01 < P < 0.05, “**” P < 0.01; “***” P < 0.001.

The results showed that:

The pain threshold can be preventatively increased by subcutaneously administering 0.1-2.5 mg/kg of DS002 every 6 days for 3 consecutive times to the paclitaxel-induced rat neuropathic pain model, and a certain dose relationship and obvious time-effect relationship is shown.

### Example 2 Pharmacodynamic Experiment of DS002 on Vincristine-Induced Neuropathic Pain Model in SD Rats

1. Establishment of Vincristine-Induced Neuropathic Pain Model in Rats

With reference to the above-mentioned experimental method and Example 1, except for the blank control group, the other groups of animals were injected intraperitoneally with a vincristine solution (0.1 mg/kg) from DO to D9, respectively, to establish a vincristine-induced neuropathic pain model in SD rats, namely, the CIPN pain model.

2. Experimental Results

2.1 Preventive Effect on Mechanical Allodynia

On day 7 (D7) after modeling, von Frey test was performed, the mean value of mechanical paw withdrawal threshold in blank control group animals was 14.63±0.28 g; the mean value of mechanical paw withdrawal threshold of vehicle control, s.c., D-3/D3/D9 group was 4.88±1.06 g, there was a significant difference compared with the Blank control group (P<0.0001), indicating that the modeling was successful. The mechanical paw withdrawal threshold of Test sample DS002, 0.1 mg/kg, s.c., D-3/D3/D9 groups was 7.02±1.05 g, and there was no significant difference compared with the vehicle control group (P>0.05). As for the test sample DS002, 0.5 mg/kg, s.c., D-3/D3/D9 group and the test sample DS002, 2.5 mg/kg, s.c., D-3/D3/D9 group, the mechanical paw withdrawal thresholds of the animals were 8.20±1.78 g and 8.43±0.92 g, respectively, and there was significant difference compared with the vehicle control group (P<0.05).

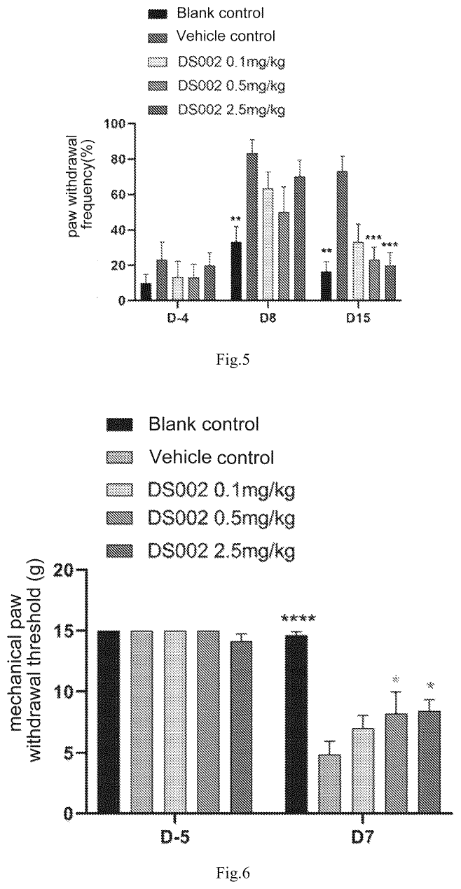

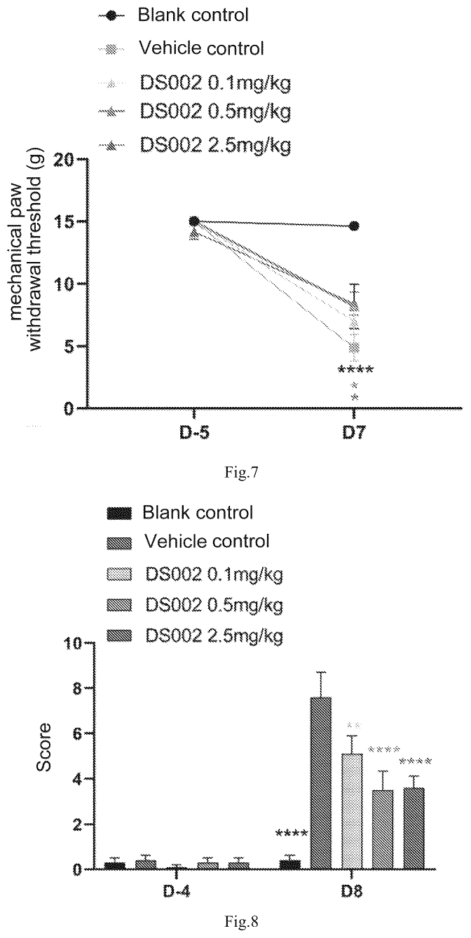

During the test, the histogram and graph of the mechanical paw withdrawal threshold of animals in each group are shown in FIGS. 6 and 7, and the mean values of the thresholds are shown in Table 3.

TABLE 3
Mechanical paw withdrawal threshold

| | | Mean value of mechanical paw withdrawal threshold | | |
|---|---|---|---|---|
| | | (g) ± S.E | | |
| | | 2020 Sep. 12 | 2020 Sep. 24 | 2020 Oct. 1 |
| Group | | D-5 | D7 | D14 |
| Blank control | Mean ± S.E | 15.00 ± 0.00 | 14.63 ± 0.28**** | 13.66 ± 0.71**** |
| Vehicle control, s.c., | Mean ± S.E | 15.00 ± 0.00 | 4.88 ± 1.06 | 4.50 ± 0.81 |
| D-3/D3/D9 | | | | |
| DS002-0.1 mg/kg, s.c., | Mean ± S.E | 15.00 ± 0.00 | 7.02 ± 1.05 | 9.76 ± 1.10**** |
| D-3/D3/09 | | | | |
| DS002-0.5 mg/kg, s.c., | Mean ± S.E | 15.00 ± 0.00 | 8.20 ± 1.78* | 11.00 ± 0.94**** |
| D-3/D3/D9 | | | | |
| DS002-2.5 mg/kg, s.c., | Mean ± S.E | 14.16 ± 0.60 | 8.43 ± 0.92* | 13.31 ± 0.76**** |
| D-3/D3/D9 | | | | |

Note: 1.
The day on formally establishing the model with vincristine administration was recorded as D0;
2. Compared with vehicle control group, “*” 0.01 < P < 0.05, “****” P < 0.0001.

The results showed that: compared with the Vehicle control, after injection of the agent DS002 of the present invention, the mechanical paw withdrawal threshold of rats was significantly increased.

2.2 Preventive Effect on Cold Allodynia

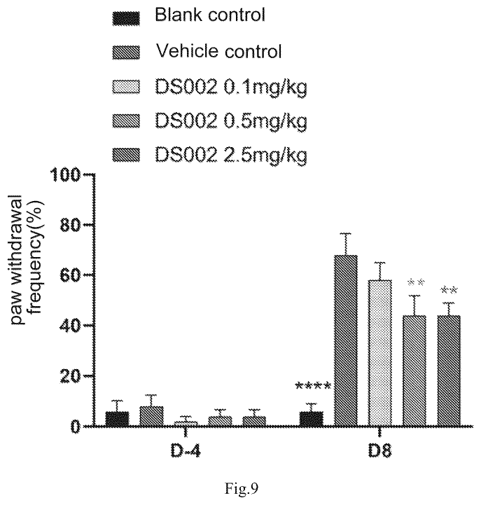

On day 8 (D8) after modeling, the acetone test was performed, the mean value of acetone-induced foot withdrawal behavior total score in blank control group animals was 0.40±0.22, and the mean value of the response frequency was 6.00±3.06%; the mean value of foot withdrawal behavior total score in vehicle control, s.c., D-3/D3/D9 group was 7.60±1.12, and there was significant difference compared with the blank control group (P<0.0001), the mean value of the response frequency was 68.00±8.54%, and there was significant difference compared with the blank control group (P<0.0001), indicating that the modeling was successful. The mean value of foot withdrawal behavior total score in the test sample DS002, 0.1 mg/kg, s.c., D-3/D3/D9 group animals was 5.10±0.80, and there was significant difference compared with the vehicle control group (P<0.01); The mean value of the response frequency was 58.00±6.96%, and there was no significant difference compared with the vehicle control group (P>0.05). As for the test sample DS002, 0.5 mg/kg, s.c., D-3/D3/D9 group and test sample DS002, 2.5 mg/kg, s.c., D-3/D3/D9 group, the mean values of foot withdrawal behavior total score of the animals were 3.50±0.83 and 3.60±0.52, respectively, and there were significant differences compared with the vehicle control group (P<0.0001); the mean values of the response frequency were 44.00±7.77% and 44.00±4.99%, respectively, and there were significant differences compared with the vehicle control group (P<0.01). The histogram of acetone-induced foot withdrawal response score and the histogram of response frequency of cold allodynia in each group animals during the test are shown in FIG. 8 and FIG. 9, and the mean values of total score and the mean values of response frequency are shown in Table 4.

TABLE 4
Mean values of acetone-induced foot withdrawal response score and the response
frequency of cold allodynia in each group of animals during the test

| | | Mean value of total score and mean value of response frequency | | | | | |
|---|---|---|---|---|---|---|---|
| | | 2020 | | 2020 | | 2020 | |
| | | Sep. 13 | | Sep. 25 | | Oct. 2 | |
| | | D-4 | | D 8 | | D 15 | |
| Group | | Score | frequency | Score | frequency | Score | frequency |
| Blank | Mean ± | 0.30 ± | 6.00 ± | 0.40 ± | 6.00 ± | 1.10 ± | 16.00 ± |
| control | S.E | 0.21 | 4.27% | 0.22**** | 3.06%**** | 0.43**** | 5.81%**** |
| Vehicle | Mean ± | 0.40 ± | 8.00 ± | 7.60 ± | 68.00 ± | 8.00 ± | 76.00 ± |
| control, | S.E | 0.22 | 4.42% | 1.12 | 8.54% | 1.31 | 9.33% |
| s.c., | | | | | | | |
| D-3/D | | | | | | | |
| 3/D 9 | | | | | | | |
| DS002-0.1 | Mean ± | 0.10 ± | 2.00 ± | 5.10 ± | 58.00 ± | 3.00 ± | 42.00 ± |
| mg/kg, | S.E | 0.10 | 2.00% | 0.80* | 6.96% | 0.49**** | 7.57%*** |
| s.c., | | | | | | | |
| D-3/D | | | | | | | |
| 3/D 9 | | | | | | | |
| DS002-0.5 | Mean ± | 0.30 ± | 4.00 ± | 3.50 ± | 44.00 ± | 2.80 ± | 34.00 ± |
| mg/kg, | S.E | 0.21 | 2.67% | 0.83**** | 7.77%* | 0.55**** | 5.21%**** |
| s.c., | | | | | | | |
| D-3/D | | | | | | | |
| 3/D 9 | | | | | | | |
| DS002-2.5 | Mean ± | 0.30 ± | 4.00 ± | 3.60 ± | 44.00 ± | 1.80 ± | 24.00 ± |
| mg/kg, | S.E | 0.21 | 2.67% | 0.52**** | 4.99%* | 0.61**** | 7.18%**** |
| s.c., | | | | | | | |
| D-3/D | | | | | | | |
| 3/D 9 | | | | | | | |

Note:
1. The day on formally establishing the model with vincristine administration was recorded as D 0;
2. Compared with vehicle control group, “*” 0.01 < P < 0.05; “**” P < 0.01; “***” P < 0.001; “*****” P < 0.0001.

### Example 3

1. Establishment of a Cisplatin-Induced Neuropathic Pain Model in Rats

With reference to the above-mentioned experimental method and Example 1, except for the blank control group, the animals of other groups were injected with a cisplatin solution (4 mg/kg) in the tail veins on DO and D6, respectively, to establish a cisplatin-induced neuropathic pain model in SD rats, namely the CIPN pain model.

2. Experimental Results

2.1 Preventive Effect on Mechanical Allodynia

The basal values of the mechanical allodynia and cold allodynia were measured on day 5 (D-5) and day 4 (D-4) before modeling. On day 3 (D-3), the normal rats were divided into 4 groups by random block method and administered according to their body weight. The average weight of animals in each group after grouping was about 250 g. As for the animals of blank control group, vehicle control, s.c., D-3/D3/D9/D16 group, test sample DS002, 0.02 mg/kg, s.c., D-3/D3/D9/D16 group and test sample DS002, 0.5 mg/kg, s.c., D-3/D3/D9/D16 group, the basal values of mechanical paw withdrawal threshold were 26.00±0.00 g, 23.95±1.63 g, 23.50±1.68 g and 22.77±1.71 g, respectively; the basal values of acetone-induced foot withdrawal behavior total score were 0.50±0.34, 0.55±0.31, 0.27±0.19 and 0.55±0.28, respectively; the basal values of the response frequency were 6.00±4.27%, 7.27±4.07%, 3.64±2.44% and 5.45±5.45±2.82%, respectively.

On day 7 (D7) after modeling, von Frey test was performed, the mean value of the mechanical paw withdrawal threshold of the animals in the blank control group was 23.20±1.41 g; the mean value of the mechanical paw withdrawal threshold of the vehicle control, s.c., D-3/D3/D9/D16 group was 8.00±1.60 g, there was significant difference compared with the blank control group (P<0.0001), indicating that the modeling was successful. As for the animals of Test sample DS002, 0.02 mg/kg, s.c., D-3/D3/D9/D16 group and Test sample DS002, 0.5 mg/kg, s.c., D-3/D3/D9/D16 group, the mechanical paw withdrawal thresholds were 8.87±1.98 g and 7.62±1.18 g respectively, and there was no significant difference compared with the vehicle control group (P>0.05).

On day 14 (D14) after modeling, von Frey test was performed, the mean value of mechanical paw withdrawal threshold of animals in blank control group was 18.87±2.17 g; The mean value of mechanical paw withdrawal threshold of vehicle control, s.c., D-3/D3/D9/D16 group was 5.32±0.89 g, and there was significantly difference compared with the blank control group (P<0.001), indicating that the modeling was successful. The mean value of mechanical paw withdrawal threshold of the animals in the test sample DS002, 0.02 mg/kg, s.c., D-3/D3/D9/D16 group was 7.22±0.77 g, and there was no significant difference compared with the vehicle control group (P>0.05); The mean value of mechanical paw withdrawal threshold of the animals in Test sample DS002, 0.5 mg/kg, s.c., D-3/D3/D9/D16 group was 10.73±1.60 g, and there was significant difference compared with the vehicle control group (P<0.05).

On day 21 (D21) after modeling, von Frey test was performed, the mean value of mechanical paw withdrawal threshold of animals in blank control group was 19.50±2.38 g; the mean value of mechanical paw withdrawal threshold of vehicle control, s.c., D-3/D3/D9/D16 group was 6.22±0.88 g, there was significant difference compared with the blank control group (P<0.001), indicating that the modeling was successful. The mean value of mechanical paw withdrawal threshold of the animals in Test sample DS002, 0.02 mg/kg, s.c., D-3/D3/D9/D16 group was 14.22±2.63 g, and there was significant difference compared with the vehicle control group (P<0.05); The mean value of mechanical paw withdrawal threshold of the animals in Test sample DS002, 0.5 mg/kg, s.c., D-3/D3/D9/D16 group was 16.37±2.36 g, there was significant difference compared with vehicle control group (P<0.01).

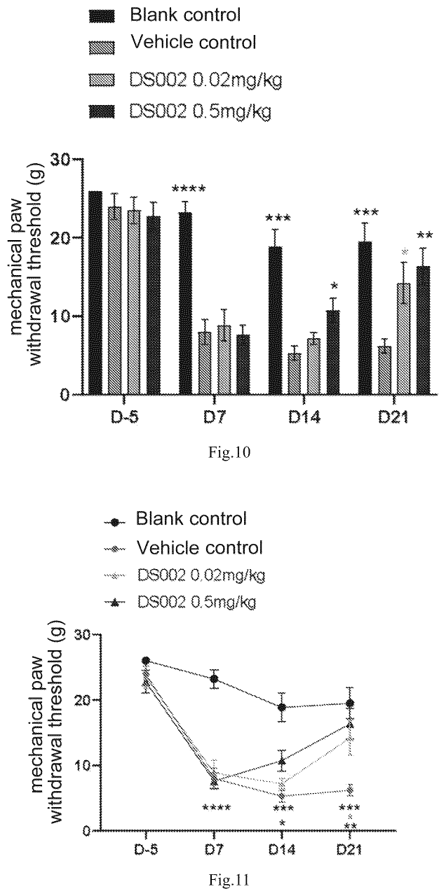

During the test, the histogram and graph of mechanical paw withdrawal threshold of animals in each group are shown in FIG. 10 (compared with the threshold of the vehicle control group, *0.01<P<0.05; **P<0.01; ***P<0.001, **** P<0.0001) and FIG. 11, the mean values of the thresholds are shown in Table 5.

TABLE 5
Mechanical paw withdrawal thresholds

| | | Mean value of mechanical paw withdrawal threshold (g) ± S.E | | | |
|---|---|---|---|---|---|
| | | 2020 Aug. 26 | 2020 Sep. 7 | 2020 Sep. 14 | 2020 Sep. 21 |
| Group | | D-5 | D7 | D14 | D21 |
| Blank control | Mean ± S.E | 26.00 ± 0.00 | 23.20 ± 1.41**** | 18.87 ± 2.17*** | 19.50 ± 2.38*** |
| Vehicle control, s.c., | Mean ± S.E | 23.95 ± 1.63 | 8.00 ± 1.60 | 5.32 ± 0.89 | 6.22 ± 0.88 |
| D- 3/D3/D9/D16 | | | | | |
| DS002, 0.02 mg/kg, | Mean ± S.E | 23.50 ± 1.68 | 8.87 ± 1.98 | 7.22 ± 0.77 | 14.22 ± 2.63* |
| s.c., D- | | | | | |
| 3/D3/D9/D16 | | | | | |
| DS002, 0.5 mg/kg, | Mean ± S.E | 22.77 ± 1.71 | 7.62 ± 1.18 | 10.73 ± 1.60* | 16.37 ± 2.36** |
| s.c., D- 3/D3/D9/D16 | | | | | |

The results showed that: compared with the vehicle control, after injection of the agent DS002 of the present invention, the mechanical paw withdrawal threshold of rats was significantly increased.

2.2 Preventive Effect on Cold Allodynia

On day 8 (D8) after modeling, the acetone test was performed, the mean value of acetone-induced foot withdrawal behavior total score of animals in blank control group was 0.70±0.42, and the mean value of the response frequency was 8.00±4.42%; the mean value of foot withdrawal behavior total score of vehicle control, s.c., D-3/D3/D9/D16 group was 0.73±0.27, and there was no significant difference compared with the blank control group (P>0.05). The mean value of the response frequency was 10.91±4.15%, and there was no significant difference compared with the blank control group (P>0.05). The mean values of foot withdrawal behavior total score of animals in Test sample DS002, 0.02 mg/kg, s.c., D-3/D3/D9/D16 group and Test sample DS002, 0.5 mg/kg, s.c., D-3/D3/D9/D16 group were 0.55±0.31 and 0.45±0.25, respectively, and there was no significant difference compared with the vehicle control group (P>0.05); the mean values of the response frequency were 7.27±4.07% and 7.27±4.07%, respectively, and there was no significant difference compared with the vehicle control group (P>0.05).

On day 15 (D15) after modeling, the acetone test was performed, the mean value of acetone-induced foot withdrawal behavior total score of animals in blank control group was 0.80±0.49, and the mean value of the response frequency was 12.00±6.11%; the mean value of foot withdrawal behavior total score of vehicle control, s.c., D-3/D3/D9/D16 group was 0.91±0.37, and there was no significant difference compared with the blank control group (P>0.05), and the mean value of the response frequency was 14.55±5.45%, and there was no significant difference compared with the blank control group (P>0.05). The mean values of foot withdrawal behavior total score of animals in Test sample DS002, 0.02 mg/kg, s.c., D-3/D3/D9/D16 group and Test sample DS002, 0.5 mg/kg, s.c., D-3/D3/D9/D16 group were 1.18±0.55 and 0.45±0.25, respectively, and there was no significant difference compared with the vehicle control group (P>0.05); the mean values of the response frequency were 16.36±5.92% and 7.27±4.07%, respectively, and there was no significant difference compared with the vehicle control group (P>0.05).

On day 22 (D22) after modeling, the acetone test was performed, the mean value of acetone-induced foot withdrawal behavior total score of animals in blank control group was 0.60±0.27, and the mean value of the response frequency was 10.00±4.47%; the mean value of foot withdrawal behavior total score of vehicle control, s.c., D-3/D3/D9/D16 group was 1.00±0.40, and there was no significant difference compared with the blank control group (P>0.05), and the mean value of the response frequency was 14.55±4.74%, and there was no significant difference compared with the blank control group (P>0.05). The mean values of foot withdrawal behavior total score of animals in Test sample DS002, 0.02 mg/kg, s.c., D-3/D3/D9/D16 group and Test sample D5002, 0.5 mg/kg, s.c., D-3/D3/D9/D16 group were 0.64±0.31 and 0.73±0.30, respectively, and there was no significant difference compared with the vehicle control group (P>0.05); the mean values of the response frequency were 9.09±4.15% and 10.91±4.15%, respectively, and there was no significant difference compared with the vehicle control group (P>0.05).

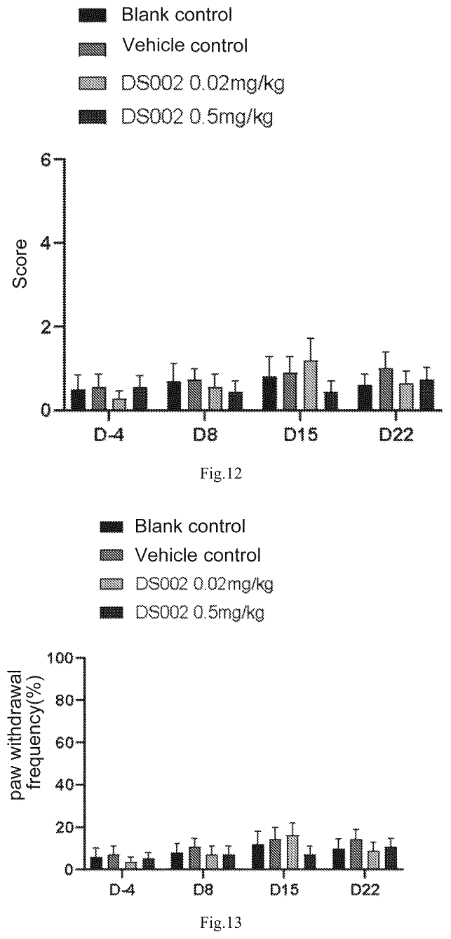

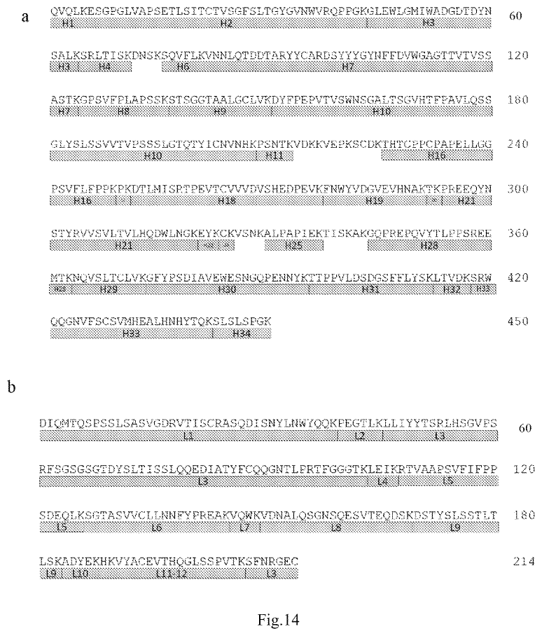

During the test, the histogram of acetone-induced foot withdrawal response score and the histogram of response frequency of cold allodynia of animals in each group are shown in FIG. 12 and FIG. 13. The mean value of total score and the mean value of the response frequency are shown in Table 6.

TABLE 6
Mean values of acetone-induced foot withdrawal response score and the response
frequency of cold allodynia in each group of animals during the test

| | | Mean value of total score and mean value of response frequency | | | | | | | |
|---|---|---|---|---|---|---|---|---|---|
| | | 2020 | | 2020 | | 2020 | | 2020 | |
| | | Aug. 26 | | Sep. 8 | | Sep. 15 | | Sep. 22 | |
| | | D-4 | | D 8 | | D 15 | | D 22 | |
| Group | | Score | frequency | Score | frequency | Score | frequency | Score | frequency |
| Blank | Mean ± | 0.50 ± | 6.00 ± | 0.70 ± | 8.00 ± | 0.80 ± | 12.00 ± | 0.60 ± | 10.00 ± |
| control | S.E | 0.34 | 4.27% | 0.42* | 4.42% | 0.49 | 6.11% | 0.27 | 4.47% |
| Vehicle | Mean ± | 0.55 ± | 7.27 ± | 0.73 ± | 10.91 ± | 0.91 ± | 14.55 ± | 1.00 ± | 14.55 ± |
| control, | S.E | 0.31 | 4.07% | 0.27 | 4.15% | 0.37 | 5.45% | 0.40 | 4.74% |
| s.c., | | | | | | | | | |
| D-3/D 3/D | | | | | | | | | |
| 9/D 16 | | | | | | | | | |
| DS002-0.02 | Mean ± | 0.27 ± | 3.64 ± | 0.55 ± | 7.27 ± | 1.18 ± | 16.36 ± | 0.64 ± | 9.09 ± |
| mg/kg, | S.E | 0.19 | 2.44% | 0.31 | 4.07% | 0.55 | 5.92% | 0.31 | 4.15% |
| s.c., | | | | | | | | | |
| D-3/D 3/D | | | | | | | | | |
| 9/D 16 | | | | | | | | | |
| DS002-0.5 | Mean ± | 0.55 ± | 5.45 ± | 0.45 ± | 7.27 ± | 0.45 ± | 7.27 ± | 0.73 ± | 10.91 ± |
| mg/kg, | S.E | 0.28 | 2.82% | 0.25 | 4.07% | 0.25 | 4.07% | 0.30 | 4.15% |
| s.c., | | | | | | | | | |
| D-3/D 3/D | | | | | | | | | |
| 9/D 16 | | | | | | | | | |

Note:
1. The day on formally establishing the model with cisplatin administration was recorded as D 0;

The results showed that, compared with the vehicle control, after the injection of the agent DS002 of the present invention, the cold pain threshold of the rats was not significantly improved.

In conclusion, the humanized recombinant monoclonal antibody targeting nerve growth factor DS002 of the present invention can significantly improve the pain threshold.

### DISCUSSION

Because pain caused by chemotherapy drugs is not caused by a single mechanism, but a result of multi-factor and multi-link interaction, common clinical analgesics such as morphine and other opioid analgesics and non-steroidal analgesics are not effective in the treatment of CIPN pain, although they have significant effects in the treatment of other clinical pains.

Duloxetine, a selective serotonin (5-HT) and norepinephrine (NE) reuptake inhibitor, is not a traditional analgesic in terms of mechanism, but it has a certain effect in the treatment of CIPN pain. Therefore, analgesics which are proved to be effective in other clinical pain indications cannot be directly applied to pain caused by CIPN, and vice versa.

Although it has been proved by preclinical and clinical data that NGF antibody has obvious curative effects in indications such as osteoarthritis and chronic low back pain, there is no report on whether such drugs can be used in CIPN pain. In the research of the present invention, the inventors unexpectedly discovered for the first time that NGF antibody has surprising curative effect on CIPN pain, and has a significant curative effect on CIPN pain ineffective or refractory to conventional analgesics, so this drug can be used as anti-CIPN pain specific medicine for the prevention and/or treatment of CIPN pain.

All documents mentioned herein are incorporated by reference in this application as if each document were individually incorporated by reference. In addition, it should be understood that after reading the above content of the present invention, those skilled in the art may make various changes or modifications to the present invention, and these equivalent forms also fall within the scope defined by the appended claims of the present application.

1. Use of a monoclonal antibody targeting nerve growth factor, wherein, it is used in the preparation of a drug for the treatment and/or prevention of chemotherapy-induced peripheral neuropathic pain, wherein the chemotherapy-induced peripheral neuropathy is caused by chemotherapeutic agents.

2. The use according to claim 1, wherein the monoclonal antibody is a humanized recombinant monoclonal antibody.

3. The use according to claim 1, wherein the heavy chain sequence and light chain sequence of the monoclonal antibody are shown in SEQ ID NO:3 and SEQ ID NO:4, respectively.

4. The use according to claim 1, wherein the chemotherapeutic agent is selected from the group consisting of taxanes, platinums, vinca alkaloids, gemcitabine, bortezomib, thalidomide, vinorelbine coast, or combinations thereof.

5. The use according to claim 1, wherein the chemotherapeutic agent is selected from the group consisting of paclitaxel, cisplatin, vincristine, or combinations thereof.

6. The use according to claim 1, wherein the monoclonal antibody targeting nerve growth factor is administered before, during or after administration of the chemotherapeutic agent.

7. The use according to claim 1, wherein the dose of the monoclonal antibody targeting nerve growth factor is 50-2000 mg/50 kg.

8. A pharmaceutical composition comprising:
i) a monoclonal antibody targeting nerve growth factor;
ii) one or more of the chemotherapeutic agents; and
iii) a pharmaceutically acceptable carrier.

9. The pharmaceutical composition according to claim 8, wherein the pharmaceutical composition is an injection.

10. A kit of a drug for treating and/or preventing chemotherapy-induced peripheral neuropathic pain, wherein the kit contains a container in which a monoclonal antibody targeting nerve growth factor or a pharmaceutical composition comprising the same is contained; and a label or instruction that indicates that the kit is used to treat and/or prevent chemotherapy-induced peripheral neuropathic pain.

11. The kit according to claim 10, further comprising a companion diagnostic reagent for detecting NGF.


SEQUENCE LISTING (4 sequences)

SEQ ID NO: 1 - protein, 117 aa, artificial sequence
  heavy chain variable region of anti-NGF monoclonal antibody
QVQLVQSGAEVKKPGASVKVSCKASGYTFTNYLIEWVRQAPGQGLEWMGVINPRSGATNYNEKFKDRLTITKDTSKNQVVLTMTNMDPVDTATYYCARGNYRFEAYWGQGTLVTVSS

SEQ ID NO: 2 - protein, 106 aa, Artificial Sequence
  light chain variable region of anti-NGF monoclonal antibody
DILMTQSQKFMSTSVGDRVSITCKASQNVRTAVAWYQQKPGQSPKALILASNRHTGVPDRFTGSGSGTDFTLTISNMQSEDLADYFCQQYSSYPFTFGSGTKLEIK

SEQ ID NO: 3 - protein, 450 aa, artificial sequence
  heavy chain of anti-NGF monoclonal antibody
QVQLKESGPGLVAPSETLSITCTVSGFSLTGYGVNWVRQPPGKGLEWLGMIWADGDTDYNSALKSRLTISKDNSKSQVFLKVNNLQTDDTARYYCARDSYYYGYNFFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 214 aa, artificial sequence
  light chain of anti-NGF monoclonal antibody
DIQMTQSPSSLSASVGDRVTISCRASQDISNYLNWYQQKPEGTLKLLIYYTSRLHSGVPSRFSGSGSGTDYSLTISSLQQEDIATYFCQQGNTLPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC